(19) United States
(12) Patent Application Publication
(10) Pub. No.: US 2007/0025960 A1
(43) Pub. Date: Feb. 1, 2007

(54) Bisphosphonates for prophylaxis and therapy against bioterrorism agents

(75) Inventors: C. David Pauza, Baltimore, MD (US); Peter Scott Evans, Baltimore, MD (US); Maria S. Salvato, Baltimore, MD (US); Ilia Tikhonov, Baltimore, MD (US)

Correspondence Address: INTELLECTUAL PROPERTY / TECHNOLOGY LAW, PO BOX 14329, RESEARCH TRIANGLE PARK, NC, 27709, US

(73) Assignee: University of Maryland Biotechnology Institute, Baltimore, MD (US)

(21) Appl. No.: 10/556,954

(22) PCT Filed: May 17, 2004
(86) PCT No.: PCT/US04/15683

§ 371 Date: Jan. 30, 2006

Related U.S. Application Data
(60) Provisional application No. 60/471,454, filed on May 16, 2003.

(51) Int. Cl. A61K 38/20; A61K 31/675; A61K 31/663; A61K 39/12; A61K 39/02

(52) U.S. Cl. 424/85.2; 514/89; 514/102; 424/234.1; 424/204.1

(57) ABSTRACT

Disclosed is a method of activating γ/δ T cells to provide temporary protection against a broad spectrum of infectious biological threats to the military and support personnel and exposed persons. γ/δ T cells are activated by administration of high doses of bisphosphonates.

### BACKGROUND OF THE INVENTION

1. Field of the Invention

The present invention relates to preventing or counteracting the effects of biochemical warfare agents, and more particularly, to administering bisphosphonates and functionally active analogs to persons exposed or potentially exposed to these agents.

2. Background of Related Art

A critical military need is to provide deployed personnel with broad protection against biological weapons in order to decrease battlefield casualties and to increase confidence that the attacks are survivable. Military and support personnel are at risk from biological weapons including smallpox, anthrax, tularemia, plague and other pathogens, in addition to genetically engineered versions of these and other pathogens that escape vaccination, resist antibiotics, and avoid detection by standard diagnostic tests. Vaccines provide an important measure of protection, provided that vaccination programs are targeted at the exact agents later used in the field and the biological weapons were not specially engineered to escape vaccine-induced immunity. For example, existing anthrax vaccines will counter one obvious threat, and likely motivate our enemies to develop more virulent strains or to explore alternate weapons strategies, and as such, further steps must be taken to protect potentially exposed military and support personnel.

Direct manipulation of the innate immune system could protect military and support personnel as part of an integrated physical and medical system for competing against and deterring the effects of biological weapons, but heretofore has not been explored. The innate immune response to infection is the body's first line of defense against pathogens and includes activation of human T lymphocytes. Human T lymphocytes include the major population expressing the alpha/beta T cell receptor and a minor population (3-10%) expressing the gamma/delta T cell receptor. The gamma/delta T cells (γ/δ) are unique compared to alpha/beta T cells, and the differences are key to their distinct roles in pathogen immunity. The distinguishing characteristic of γ/δ T cells is their receptors, surface proteins that bind to molecules and trigger the cells into action, and consist of small proteins called gamma and delta chains.

Studies of γ/δ T cells suggest that these T cells fight off bacteria, such as those that cause tuberculosis, as well as viruses. Gamma/delta T cells exposed to compounds emitted from bacteria or viruses produce high levels of cytokines including TNF-α and IFNγ, chemokines including RANTES, MP-1α, MIP-1β,—and Lymphotactin (Cipriani et al., 2000), and human neutrophil proteins or defensins (Batoni et al., 1998). Activated γ/δ T cells proliferate rapidly and can increase from around 5% to more than 70% of peripheral blood T cells during the convalescent phase of infections such as tularemia (Kroca, Tarnvik, and Sjostedt, 2000; Sumida et al., 1992), brucellosis (Bertotto et al., 1993), or plague (Williamson et al., 2000). Activated γ/δ T cells also become cytotoxic for infected cells. They are known to efficiently lyse cells infected with several viruses including HIV, hepatitis, and vaccinia (Sciammas and Bluestone, 1998; Sciammas and Bluestone, 1999; Sciammas et al., 1994; Sciammas et al., 1997). Clearly, our nation's military and support personnel require broad biological protection, to complement their physical barrier prevention equipment. A slower disease onset would increase the time available to accurately identify the pathogen, to apply specific treatments, and to utilize post-exposure vaccination as appropriate. Heretofore, stimulation of the gamma/delta T cells to bolsters overall immunity has not been used to prevent or counteract the effects of biological weapons. Accordingly, it is highly relevant to develop a composition and program that will stimulate the gamma/delta T cells thereby increasing resistance to a broad spectrum of biochemical warfare agents including both recognized and potentially novel threats.

### SUMMARY OF THE INVENTION

An ideal target for new methods to protect against infectious biological weapons is the gamma/delta T cells present in all healthy individuals. The unique mechanism for γ/δ T cell activation shows that these cells can be manipulated to increase resistance to infectious diseases. The identified subset of γ/δ T cells, expressing the Vγ2/Vδ2 T cell receptor, is activated in all of the disease examples mentioned herein, in addition to a large number of other bacterial and viral infections (Bukowski, Morita, and Brenner, 1999). The activated γ/δ T cells recognize stress molecules expressed on the cell surface, and then marking infected cells as targets for γ/δ T cell-mediated cytotoxicity. This is one mechanism for γ/δ T cell resistance to infectious diseases. A second mechanism involves immunoregulatory functions of activated γ/δ T cells, to recruit other immune cell types to the site of infection (through the action of secreted chemokines) and to promote protective immune responses (through the action of cytokines including TNF-α and IFNγ). These effects of γ/δ T cells define this cell type as a potent effector of immunity to a broad spectrurn of infectious diseases. Importantly, this same subset of γ/δ T cells respond to allypyrophosphate, alkylamine, and bisphosphonate (BP) compounds, and all stimuli elicit qualitatively identical responses, including cytokine/chemokine secretion and increased cytotoxicity.

Thus, one aspect of the present invention is to identify orally available compounds that can be self-administered and that activate gamma/delta T cells to provide protection against infectious biological weapons.

Another aspect of the present invention provides for a method of stimulating γ/δ T cells to provide protection against infectious biological weapons, the method comprising administering a therapeutically effective amount of a bisphosphonate or salt thereof, wherein a therapeutically effective amount is an amount that stimulates γ/δ T cells, thereby inducing secretion of chemokines and cytokines.

Yet another aspect of the present invention provides for a method of protecting a person from disease arising from exposure to a biological pathogen comprising administering a composition comprising an effective amount of at least one bisphosphonate sufficient to stimulate γ/δ T cells. The bisphosphonate, alone or in combination with a pharmaceutically effective carrier, may be administered by an oral, intravenous, or subcutaneous route. The bisphosphonate may be selected from the group consisting of risedronate, alendronate, zoledronic acid, cimadronate, clodronate, tiludronate, etidronate, ibandronate, piridronate and pamidronate. Preferably, the composition comprises at least 30 mg of a bisphosphonate, and more preferably from about 60 to about 120 mg of a bisphosphonate. Most preferably, the composition comprises risedronate.

Another aspect of the present invention provides for a method of protecting a person against infection after exposure to a biological pathogen comprising adrministering an effective amount of bisphosphonate immediately after said exposure

Further, the present invention provides for a method of protecting a person prior to coming into contact with a biological pathogen, the method comprising administering an effective amount of bisphosphonate immediately prior to coming into contact with a biological pathogen.

The composition comprising a bisphosphonate may further comprise a vaccine effective against biological warfare agents. Several vaccines have been developed against likely bioterrorist agents. Given the present state of development for these vaccines and other factors, it seems unlikely that a large panel of preventive vaccines will be administered to military and support personnel. Rather, there is a need for strategies that maximize the value of new vaccine products while limiting the risk and complications that come with introducing a new menu of mandatory vaccines. One approach to this issue is to utilize post-exposure vaccination whenever possible. Under this scenario, vaccines are only administered to individuals with a confirmed risk because of pathogen exposure during a biological weapons attack. Thus, prophylaxis with risedronate or another γ/δ T cell activating compound may be coupled with post-exposure vaccination as an integrated strategy.

Other features and advantages of the invention will be apparent from the following detailed description and claims.

### DETAILED DESCRIPTION OF THE INVENTION

As defined herein, a “bisphosphonate” includes any compound which is an analog of endogenous pyrophosphate whereby the central oxygen is replaced by carbon. Bisphosphonates include aminobisphosphonates. Bisphosphonates include, but are not limited to the compounds zoledronic acid, risedronate, alendronate, cimadronate, clodronate, tiludronate, etidronate, ibandronate, piridronate or pamidronate. Examples of pharmaceutically acceptable salts of the compounds include salts derived from an appropriate base, such as an alkali metal (for example, sodium, potassium), an alkaline earth metal (for example, calcium, magnesium), ammonium and NR′₄⁺ (wherein R′ is C₁-C₄ alkyl). Pharmaceutically acceptable salts of an amino group include salts of: organic carboxylic acids such as acetic, lactic, tartaric, malic, lactobionic, fumaric, and succinic acids; organic sulfonic acids such as methanesulfonic, ethanesulfonic, isethionic, benzenesulfonic and p-toluenesulfonic acids; and inorganic acids such as hydrochloric, hydrobromic, sulfuric, phosphoric and sulfamic acids. Pharmaceutically acceptable salts of a compound having a hydroxyl group consist of the anion of said compound in combination with a suitable cation such as Na⁺, NH₄⁺, or NR′₄⁺ (wherein R′ is for example a C₁₋₄ alkyl group).

As defined herein, the word “treatment” refers to either (i) the prevention of infection or reinfection (prophylaxis), or (ii) the reduction or elimination of symptoms of the disease of interest (therapy).

As defined herein, “stimulation of γ/δ T cell functions” means an increased cell proliferation, production of cytokines, production of chemokines or cytotoxicity for infected cells.

As defined herein, a “high-dose of bisphosphonate” is a one-dose amount greater than that used to inhibit bone resorption or in the treatment of Paget's disease. A high-dose amount of bisphosphonate is sufficient to adequately stimulate γ/δ T cells. Such a high dose amount may be 2-3 times greater than the dose required to inhibit bone resorption. High dose of bisphosphonate may range from about 40 mg to about 200 mg, and preferably from about 60 mg to about 120 mg.

As defined herein, “biological weapons or pathogens” include, but are not limited to, smallpox, anthrax, tularemia and plague. Protection against other pathogens which involve γ/δ T cells include, but are not limited to, listeria, brucellosis, hepatitis, vaccinia, mycobacteria and coxsackievirus. Other diseases resulting from exposure to biological pathogens include, but are not limited to, tuberculosis, malaria, erhlichosis and bacterial meningitis.

As defined herein, “prophylactic” treatment is a treatment administered to a subject who does not exhibit signs of a disease or who exhibits early signs of the disease for the purpose of decreasing the risk of developing pathology associated with the disease.

As defined herein, “therapeutic” means a treatment administered to a subject who exhibits signs of pathology for the purpose of diminishing or eliminating those signs.

The term “therapeutically effective amount,” as used herein means an amount of a bisphosphonate compound that is sufficient to provide a beneficial effect to the subject to which the compound is administered. A beneficial effect means stimulating gamma/delta T cells.

The term “functionally active analog,” means compounds derived from a particular parent compound by straightforward substitutions that do not result in a substantial (i.e. more than 100×) loss in the biological activity of the parent compound, where such substitutions are modifications well-known to those skilled in the art, e.g., esterification, replacement of hydrogen by halogen, replacement of alkoxy by alkyl, replacement of allcyl by alkoxy, etc.

Bisphosphonate drugs are used to enhance natural immunity by increasing the activity of gamma/delta T cells. Bisphosphonates are clinically approved agents and familiar parts of the pharmacopeia as they are used frequently to treat bone demineralization disorders and hypercalcemia. Bisphosphonates are analogues of endogenous pyrophosphate whereby the central oxygen is replaced by carbon. The substituted carbon makes bisphosphonates resistant to hydrolysis, and allows two additional chains with variable structure. Due to the heightened activity of gamma/delta T cells and their capacity to resist a broad spectrum of infectious diseases, the time between exposure and disease following a biological weapons exposure is increased. This treatment attenuates a broad spectrum of pathogens, provides the critical window of opportunity needed to obtain an accurate diagnosis, initiate a treatment strategy, and possibly administer a post-exposure vaccine. The treatmnent is preferably orally delivered and self-administered.

Aminobisphosphonates and halohydrin derivatives of bisphosphonates are also suitable for use in the present invention. The aminobisphosphonates have few adverse effects and are used widely to treat bone demineralization disorders where they are generally administered at low, chronic doses. Higher, single doses, accordingly to the present invention, are used for increasing gamma/delta T cell activity and providing broad protection against biological weapons.

Alendronate (FOSAMAX) is a one example of a commonly prescribed, oral drug for osteoporosis. The indication for alendronate is bone demineralization disorder, and the drug is used in a treatment or prevention mode. Adverse reactions include esophagitis; this limits the dose and requires careful administration with the subject sitting upright and taking water. Alendronate is highly effective against bone demineralization despite the need for chronic low dosing because the drug deposits in the bone, where it inhibits osteoclast activity (Physician's Desk Reference).

Pamidronate is another BP that may be used in the present invention and is given by intravenous infusion to control hypercalcernia in the context of myeloma. In some cases, especially for pamidronate treatment, clinical data show an increase in γ/δ T cell count after drug infusion (Kunzmann, Baner, and Wilhelm, 1999).

Risedronate is highly active for stimulating γ/δ T cells and is reported to have few adverse reactions in human subjects. Risedronate use is indicated for treatment of osteoporosis (both post-menopausal and glucocorticoid-induced), and for the management of patients with Paget's disease (Nussbaum et al., 1993). For Paget's disease, the recommended dose is 30 mg/day, taken for 84 days. This dose decreased the severity of fractures and increased bone mineralization. The adverse effects were most commonly minor GI disturbances, headaches, and arthralgias. Further, risedronate is sufficiently active and has such a favorable toxicity profile, that patients might take the drug once per year and still have effective treatment for osteoporosis. Risedronate was developed specifically to reduce GI toxicity and is highly active for stimulating γ/δ T cells. Additionally, risedronate may be administered with IL-2 to achieve even more potent activation of peripheral blood T cells.

A single oral treatment of bisphosphonate can increase gamma/delta T cells activity for 30-45 days. Thus, the consequence of activating gamma/delta T cells will be to increase the interval between exposure to a biological weapons agent and the onset of disease. This allows more time for accurate diagnosis, selecting the appropriate treatment regimen, and administering post-exposure vaccines when appropriate. Alterations to the drug formulation can increase effectiveness; these alterations include repeated dosing or the addition of co-factors to increase the potency

Administration of bisphosphonate for γ/δ T cell activation may be co-administered with vaccines against Class A agents including smallpox, anthrax, tularemia and plague. Activation of γ/δ T cells increases the immune response to most vaccines. There is a rising interest in natural adjuvants for vaccines, including the use of the attenuated BCG vaccine that is used to prevent tuberculosis. BCG is a powerful activator of γ/δ T cells, and part of its effect as an adjuvant for other vaccines (Ota et al., 2002) may reflect the consequence of broad γ/δ T cell activation.

The bisphosphonates of the present invention can be formulated into therapeutic compositions in a variety of dosage forms such as, but not limited to, liquid solutions or suspensions, tablets, pills, powders, suppositories, polymeric microcapsules or microvesicles, liposomes, and injectable or infusible solutions. The preferred form depends upon the mode of administration. The compositions also preferably include pharmaceutically acceptable vehicles, carriers or adjuvants, well known in the art, such as human serum albumin, ion exchangers, alumina, lecithin, buffer substances such as phosphates, glycine, sorbic acid, potassium sorbate, and salts or electrolytes such as protamine sulfate. Suitable vehicles are, for example, water, saline, dextrose, glycerol, ethanol, or the like, and combinations thereof. Actual methods of preparing such compositions are known, or will be apparent, to those skilled in the art. See, e.g., Remington's Pharmaceutical Sciences, Mack Publishing Company, Easton, Pa., 18th edition, 1990.

The above compositions can be administered in therapeutically effective doses using conventional modes of delivery including, but not limited to, intravenous, intraperitoneal, oral, intralymphatic, or subcutaneous administration. Therapeutically effective doses will be easily determined by one of skill in the art and will depend on time of administration and/or exposure of a person to a bioterrism agent, the severity and course of the infectious agent disease, the patient's health and response to treatment, and the judgment of the treating physician.

Below are examples of specific embodiments for carrying out the present invention. The examples are offered for illustrative purposes only, and are not intended to limit the scope of the present invention in any way.

### EXAMPLE 1

Healthy, specific pathogen-free cynomolgus macaques are treated with oral risedronate in a dose ranging study. The minimum dose is equivalent to the recommended dose for healthy adult human beings, adjusted for body weight. The dose is increased by factors of 5 until systemic toxicity is encountered or a suitable level of γ/δ T cell activation is achieved.

With defined optimum dosing, the kinetics of γ/δ T cell activation are studied. These studies evaluate the change in circulating γ/δ T cell count, the duration and magnitude of cytokine/chemokine secretion, and the pattern of activation marker expression, with a goal here to define the duration of γ/δ T cell activation following a single dose of risedronate.

The minimum allowable interval between risedronate administrations is determined by repeated dosing. Normal regulation of γ/δ T cell activity involves a Fas-mediated process of cellular apoptosis (Li et al., 1998). This mechanism limits the extent of γ/δ T cell proliferation, and is similar to the homeostatic regulation of all T cell populations. Once the duration of γ/δ T cell activation following a single risedronate dose is defined, repeating dosing within and outside of the defined interval provides the minimum interval between repeat risedronate doses.

Risedronate doses start at 30 mg (given once) and increase two-fold to 120 mg given once. Adverse reactions are treated immediately. The GI tract erosion that is a common adverse effect of BP treatment, is associated with chronic drug intake and should not be a factor in single dosing. The single dose approach is used to achieve a sufficiently high blood concentration of risedronate to activate γ/δ T cells. With chronic, low level administration, it is expected that the compound would mainly load the bone reservoir and would not reach the blood levels necessary to activate the targeted lymphocyte compartment.

Little variation in γ/δ T cell numbers or phenotype in healthy individuals is expected with risedronate. In one healthy volunteer studied for more than 5 years in our laboratory, the Vδ2 subset varied between 5 and 7% of peripheral blood CD3+ cells, and the response to in vitro stimulation is nearly identical over that time. Further, molecular analysis of the Vδ2 T cell receptor repertoire in that individual revealed a surprisingly stable spectratype, a measure of diversity in the Vδ2 T cell receptor population.

γ/δ T cell responses are characterized in terms of molecular changes in the repertoire. (Evans et al., 2001; Rakasz et al., 2000a) and the profile of cytokine/chemokine expression (Wallace et al., 1996, Wallace et al., 1997b). These assays are critical for understanding the effects of BP treatment in human beings, and are also essential for validating the in vitro screening assays used to select BP or other γ/δ T cell-stimulating drugs.

### EXAMPLE 2

Animals, blood drawing and drug dosing: Twelve captive-bred, juvenile cynomolgus macaques (Macaca fasicularis) obtained as specific pathogen-free animals from a commercial breeder, and housed in the Institute of Human Virology Animal Care Facility are used. Macaques are anesthetized prior to all blood draws or examinations outside the cage. Risedronate tablets are crushed and mixed into a spread containing maple syrup and peanut butter, that is lathered on bread slices and given as the first feeding prior to regular chow. In order to condition macaques, they are given lathered bread as the first feeding every Monday morning beginning at least two weeks before the first drug dose. Any unused bread pieces are collected to account for missed drug doses. This type of drug delivery in macaques is a reliable and simple method that avoids undue stress for the animals.

### EXAMPLE 3

Treatment and blood drawing: Three blood samples are collected prior to the first treatment for baseline values. Blood is collected in EDTA tubes and is separated into plasma and mononuclear cell fractions. Cells are stored as frozen viable material at −130° C.; plasma samples are stored at −80° C. Animals are treated with drug, and blood is collected weekly for 2 weeks, then bi-weekly for 8 weeks. The animals are then rested for 4 weeks before beginning a new treatment cycle, for a total of 14 weeks for each iteration of the testing program. In addition to prebleed samples, this gives 6 experimental samples per animal. Because of the small size of cynomolgus macaques, only 7 ml samples are collected at each weekly interval.

### EXAMPLE 4

Laboratory assays: The schedule for laboratory assays is shown below in Table 1. The experimental assays are described after the table.

TABLE 1

| | Pre-3 | Pre-2 | Pre-1 | Dose | Post-1 day | Post-2 day | Post-4 day | Post-6 day | Post-8 day |
|---|---|---|---|---|---|---|---|---|---|
| Blood Draw | X | X | X | X | X | X | X | X | X |
| ELISPOT | | | X | | X | X | X | | X |
| γ/δ Proliferation | | | | | X | X | | X | X |
| Flow cytometry | | | | | X | X | X | | X |
| CBC | X | X | X | X | X | X | | | |
| Clinical Chemistry | X | X | X | X | | X | | X | |

The ELISPOT assay for γ/δ T cells was developed by the inventors for rapid assessment of γ/δ T cell function. ELISPOT measures the secretion of IFNγ, a cytokine produced by stimulated Vγ2/Vδ2 T cells. In control experiments, PBMC were stimulated with Alendronate and then measured the number of IFNγ-secreting cells at 24 hours after stimulation. The unstimulated samples showed that <0.05% of PBMC secreted IFNγ at 24 hours. The stimulated samples showed that between 5-10% of the total CD3+ T cells produced IFNγ by 24 hours and this number was equivalent to the number of Vγ2/Vδ2+ cells determined by flow cytometry. Intracellular cytokine staining assays was used to show that intracellular IFNγ at 24 hours after stimulation was restricted to the Vγ2/Vδ2 subset. In preliminary studies with peripheral blood from macaques immunized with the BCG vaccine against tuberculosis(that is known to activated macaque γ/δ T cells, Shen et al., 2002), it was shown that more than 50% of the total Vγ2/Vγ2 T cells secreted IFNγ by 24 hours in the absence of additional stimulation. For the experiments described here, the frequency of IFN γ-secreting cells in PBMC is measured with and without in vitro BP stimulation. Animals with stimulated γ/δ T cells as a result of oral risedronate treatment will already have the activating, cytokine secreting phenotype. The extent to which peripheral blood γ/δ T cells secrete IFNγ in the absence of additional in vitro BP stimulation will measure the state of cellular activation in vivo.

γ/δ T cell proliferation: The most common assay for γ/δ T cell activity is cell proliferation in response to phosphoantigens. This assay is used to confirm the results from ELISPOT assays. PBMC are assayed by flow cytometry to determine the percentage of Vδ2+ T cells. Then, the cells are stimulated with BP and cultured in IL-2 as we described previously (Enders et al., 2003; Evans et al., 2001). 10 days later, the cells are harvested and assayed again by flow cytometry. The absolute Vδ2+ T cell count is calculated before and after the 10 day culture, and cell counts are compared in cultures with and without BP. This allows for calculation a stimulation index. For PBMC from healthy individuals, a stimulation indices from 5 to 30 for BP treatment is expected, with a higher stimulation indices in in vivo.

Flow cytometry: Flow cytometry is performed to measure lymphocyte subsets and to characterize cellular phenotypes in terms of activation marker expression Dykhuizen et al., 2000). Lymphocyte subset measurements examine the CD4 and CD8 T cell subsets (alpha/beta T cells as γ/δ T cells are double negative), the CD20 B cells, the Vγ2+ and Vδ1+γ/δ T cell subsets. The standard lymphocyte subpopulation panels are part of the safety data and will show whether ABP treatment alters cell subsets other than for the γ/δ T cell targets. The Vδ1 and Vδ2 subset measurements follow the effects of ABP treatment. It will be shown that there is an increased numbers of Vδ2 cells while the Vδ1 subset remains unchanged.

In addition to measuring the subset distributions, the increase in cell surface activation markers on the Vδ2+γ/δ T cells is measured. Since the majority of γ/δ T cells in blood have a resting memory phenotype, the common T cell activation markers including CD62L and CD45RO, are not useful. Rather, CD25, CD27, CD69 and HLA-DR are used to measure γ/δ T cell activation. The changes in Vδ2 T cell expression of these activation markers after BP treatment are followed, and compared with the phenotype of Vδ1 and other T cell subsets to show that the effect is specific, and is dose dependent.

Acronym and symbol definitions

| | |
|---|---|
| BP: | bisphosphonate |
| BCG: | Bacille Calmatte-Guerin |
| CBC; | complete blood count |
| CD: | cell determinant |
| DPG: | diphosphoglycerate |
| EDTA: | ethylenediaminetetra acetic acid |
| ELISPOT: | enzyme linked immunoassay for secreting cells |
| γ/δ T cells: | gamma/delta T cells |
| GI: | gastrointestinal |
| HC V: | hepatitis C virus |
| HLA-DR: | histocompatibility locus - DR |
| IFN-γ: | Interferon gamma |
| IL-2: | INTERLEUKIN-2 |
| IND: | investigational new drug |
| M: | Mycobacterium |
| Mg: | milligram |
| MIP-1α: | Macrophage inflammatory protein 1 alpha |
| MIP-1β: | Macrophage inflammatory protein 1 beta |
| PBMC: | peripheral blood mononuclear cells |
| RANTES: | Regulated upon activation normal T cell expressed |
| | and secreted |
| SCID: | severe combined immunodeficiency disease |
| SHIV: | simian/human immunodeficiency virus |
| SIV: | simian immunodeficiency virus |
| TNF-a: | Tumor necrosis factor - alpha |
| Vδ: | V delta |
| Vγ: | V gamma |

### REFERENCES

### All Publications Cited Herein Are Hereby Incorporated by Reference in Their Entirety

Batoni, G., Esin, S., Harris, R. A., Kallenius, G. Svenson, S. B., Anderson, R., Campa, M., and Wigzell, H (1998). Gammadelta+ and CD4+alphabeta+human T cell subset responses upon stimulation with various Mycobacterium tuberculosis soluble extracts. Clinical & Experimental Immunology 112(1), 52-62.
Bertotto, A., Gerli, R., Spinozzi, F., Muscat, C., Scalise, F., Castellucci, G., Sposito, M., Candio, F., and Vaccaro, R. (1993). Lymphocytes bearing the gamma delta T cell receptor in acute Brucella meliteiisis infection. Eur J Immunolgy 23 (1177-1180).
Boom, W. H. (1996). The role of T-cell subsets in Mycobacterium tuberculosis infection. Infectious Agents & Disease 5(2), 73-81.
Bukowski, J. F., Morita, C. T., and Brenner, M. (1999). Human gamma delta T cells recognize alkylamines derived from microbes, edible plants, and tea: implications for innate immunity. Immunity 11, 57-65.
Burk, M. R., Carena, I., Donda, A., Mariani, F., Mori, L., and De Libero, G. (1997). Functional inactivation in the whole population of human V gamma 9/V delta 2 T lymphocytes induced by a nonpeptidic antagonist. Journal of Experimental Medicine 185(1), 91-7.
Cipriani, B., Borsellino, G., Poccia, F., Placido, R., Tramonti, D., Bach, S., Battistini, L., and Brosnan, C. F. (2000). Activation of C—C beta-chemokines in human peripheral blood gammadelta T cells by isopentenyl pyrophosphate and regulation by cytokines. Blood 95(1), 39-47.
Das, H., Wang, L., Kamath, A., and Bukowski, J. F. (2001). Vgamma2Vdelta T-cell receptor-mediated recognition of aminobisphosphonates. Blood 98(5), 1616-1618.
Dieli, F., Troye-Blomberg, M., Farouk, S. F., Sirecil, G., and Salerno, A. (2001). Biology of gammadelta T cells in tuberculosis and malaria. Cur Mol Med 1(4), 437-46.
Duarte, R., Kindlelan, J. M., Carracedo, J., Sanchez-Guijo, P., and Ramirez, R. (1997). Mycobacterium tuberculosis induces apoptosis in gamma/delta T lymphocytes from patients with advanced clinical forms of active tuberculosis. Clinical & Diagnostic Laboratory Immunology 4(1), 14-8.
Dykhuizen, M., Ceman, J., Mitchen, J., Zayas, M., MacDougall, A., Helgeland, J., Rakasz, E., and Pauza, C. D. (2000). Value of the CD3 marker for evaluating age-related changes in rhesus macaque CD4/CD8 T-cell ratios. Cytomtrry 40, 69-75.
Enders, P., Yin, C., Martini, F., Evans, P., Propp, N., Poccia, F., and Pauza, C. (2003). Disruption of the Vγ2 T cell receptor repertoire during HIV infection. AIDS Research and Human Retroviruses in press.
Evans, P. S., Enders, P. J., Yin, C., Ruckwardt, T., Malkovsky, M., and Pauza, C. D. (2001). In vitro stimulation with a non-peptidic alkylphosphate antigen expands cells expressing the Vγ2-Jγ1.2/Vδ2 T cell receptors. Immunology 104, 19-27.

Gan, Y.-H., Pauza, C. D., and Malkovsky, M. (1995). γδ T cells in rhesus monkeys and their response to simian immunodeficiency virus infection. Clin. Exptl Immunol. 102, 251-255.
Kroca, M., Tarnvik, A., and Sjostedt, A. (2000). The proportion of circulating gammadelta T cells increases after the first week of onset of tularaemia and remains elevated for more than a year. Clin Exp Immunology 120(2), 280-4.
Kunzmann, V., Bauer, E., and Wilhelm, M (1999). Gamma/delta T-cell stimulation by pamidronate. N Eng J Med 340(9), 737-8.
Li, B., Bassiri, H., Rossman, M D., Kramer, P., Eyuboglu, A. F., Torres, M., Sada, E., Imir, T., and Carding, S. R. (1998). Involvement of the Fas/Fas ligand pathway in activation-induced cell death of mycobacteria-reactive human gamma delta T cells: a mechanism for the loss of gamma delta T cells in patients with pulmonary tuberculosis. Journal of Immunology 161(3), 1558-67.
MacDougall, A. V., Enders, P., Hatfield, G., Pauza, D.C., and Rakasz, E. (2000). Vgamma2 TCR Repertoire Overlap in Different Anatomical Compartments of Healthy, Unrelated Rhesus Macaques. J. Immunol. 4, 2296-2302.

Martini, F., Poccia, F., GoLetti, D., Carrara, S., Vincenti, D., D'Offizi, G., Agrati, C., Ippolito, G., Colizzi, V., Pucillo, L. P., and Montesano, C. (2002). Acute human immunodeficiency virus replication causes a rapid and persistent impairment of Vgamma9Vdelta2 T cells in chronically infected patients undergoing structured treatment interruption. J Infect Dis. 186(6), 847-50.
Martini, F., Urso, R., Gioia, C., De Felici, A., Narciso, P., Amendola, A., Paglia, M. G., Colizzi, V., and Poccia, F. (2000). gammadelta T-cell anergy in human imnunodeficieney virus-infected persons with opportunistic infections and recovery after highly active antiretroviral therapy. Immunology 100(4), 481-6.
MeVay, L. D., and Carding, S. R. (1999). Generation of human γδ T cell repertoires. Critical Reviews in Immunology 19, 431-460.

Morita, C. T., Beckman, F. M., Bukowski, J. F., Tanaka, Y., Band, H., Bloom, B. R., Golan, D. E., and Brenner, M. B. (1995). Direct presentation of nonpeptide prenyl pyrosphate antigens to human γδ T cells. Immunity 3, 496-507.
Morita, C. T., Lee, H. K., Wang, H., Li, H., Mariuzza, R. A., and Tanaka, Y. (2001). Structural features of nonpeptide prenyl pyrophosphates that determine their antigenicity for human gamma delta T cells. JImmunol 167(1), 36-41.
Nussbaum, S. R., Younger, J., Vandepol, C. J., Gagel, R. F., Zubler, M. A., Chapman, R., Henderson, I C., and Mallerte, L. E. (1993). Single-dose intravenous therapy with pamidronate for the treatment of hypercalcemia of malignancy: comparison of 30-, 60-, and 90-mg dosages. Am J Med 95(3), 297-304.
Ota, M. 0., Vekemans, J., Schlegel-Haueter, S. E., Fielding, K., Sanneh, M., Kidd, M., Newport, M. J., Aaby, P., Whittle, H., Lambert, P. H., McAdam, K. P., Siegrist, C. A., and Marchant, A. (2002). Influence of Mycobacterium bovis bacillus Calmette-Guerin on antibody and cylokine responses to human neonatal vaccination. J Immunol 168(2), 919-25.
Parker, C. M., Groh, V., Band, H., Porcelli, S. A., Morita, C., Fabbi, M., Glass, D., Strominger, J. L., and Brenner, M. B (1990). Evidence for extrathymic changes in the T cell repertoire gamma/delta repertoire. J Exp Med 171, 1597-1612.
Pietschinann, P., Stohlawetz, P., Brosch, S., Steiner, G., Smolen, J. S., and Peterlik, M. (1998). The effect of alendronate on cytokine production, adhesion molecule expression, and transendothelial migration of human peripheral blood mononuclear cells. Calcif Tissue Int 63(4), 325-30.
Poquet, Y., Halary, F., Champagne, E., Davodeau, F., Gougeon, M. L., Bonneville, M., and Fournie, J. J. (1996). Human gamma delta T cells in tuberculosis. Research in immunology 147(8-9), 542-9.
Rakasz, E., MacDougall, A. V., Zayas, M. T., Ruckwardt, T., Evans, P. S., Emerson, C. L., Acker, L., Shore, M. D., Mitchen, J., and Pauza, C. D. (2000a). Characteristic TCRGV9 CDR3 length profile of rhesus macaques in PBL and colonic mucosa. Immunol. Letters 69, 171-172.
Rakasz, E., Mueller, A., Perlman, S., and Lynch, R. G. (1999). Gammadelta T cell response induced by vaginal Herpes simplex 2 infection. Immunol Lett 70(2), 89-93.
Rakasz, E. G., McDougall, A., Zayas, M., Helgeland, J. L., Ruckwardt, T., Hatfield, G., Dykhuizen, M., Mitchen, J. L., Evans, P. S., and Pauza, C. D. (2000b). γδ T cell receptor repertoire in blood and colonic mucosa of Rhesus macaques. J Med Primatol 29, 387-396.
Rock, E. P., Sibbald, P. K., Davis, M. M., and Chien, Y.-H. (1994). CDR3 length in antigen-specific immune receptors. J Exp Med 179, 323-328.
Sciammas, R., and Bluestone, J. A. (1998). HSV-1 glycoprotein I-reactive TCR gamma delta cells directly recognize the peptide backbone in a conformationally dependent manner. J Immunol 161(10), 5187-92.
Sciammas, R., and Bluestone, J. A. (1999). TCR gammadelta cells and viruses. Microbes Infect 1(3), 203-12.
Sciammas, R., Johnson, R. M., Sperling, A. I., Brady, W., Linsley, P. S., Spear, P. G., Fitch, F. W., and Bluestone, J. A. (1994). Unique antigen recognition by a herpesvirus-specific TCR-gamma delta cell. J immunol 152, 5392-5397.
Sciammas, R., Kodukula, P., Tang, Q., Hendricks, R. L., and Bluestone, J. A. (1997). T cell receptor-gamma1delta cells protect mice from herpes simplex virus type 1-induced lethal encephalitis. J Exp Med 185(11), 1969-75.
Shen, Y., Zhou, D., Qiu, L., Lai, X., Simon, M., Shen, L., Kou, Z., Wang, Q., Jiang, L., Estep, J., Hunt, R., Clagett, M., Sehgal, P. K., Li, Y., Zeng, X., Morita, C. T., Brenner, M. B., Letvin, N. L., and Chen, Z. W. (2002). Adaptive immune response of Vgamma2Vdelta2+ T cells during mycobacterial infections. Science 295 (5563), 2255-8.
Sumida, T., Maeda, T., Takahashi, H., Yoshida, S., Yonaha, F., Sakamoto, A., Tomioka, H., Koike, T., and Yoshida, S. (1992). Predominant expansion of Vgamma9/Vdelta2 T cells in tularemia patient. Infect Immun 60, 2554-2558.
Wallace, M., Bartz, S. R., Chang, W.-L., MacKenzie, D., Pauza, C. D., and Malkovsky. M. (1996). γ/δ T lymphocyte responses to human immunodeficiency virus. Clin. Exptl. Immunol 103, 177-184.
Wallace, M., Scharko, A. M., Pauza, C. D., Fisch, P., Imaoka, K., Kawabata, S., Fujihashi, K.,Kiyono, H., Tanaka, Y., Bloom, B. R., and Malkovsky, M. (1997a). Deficit in the constitutive antiturnor and nonpeptide antigen responses of˜o T cells associated with HIV infections. Abstracts for Keystone Symposium: Cellular Immunology and the Immunotherapy of Cancer-III Feb. 1-7, 1997.
Wallace, M., Scharko, A. M., Pauza, C. D., Fisch, P., Imaoka, K., Kawabata, S., Fujihashi, K., Kiyono, H., Tanaka, Y., Bloom, B. R., and Malkovsky, M. 1997b). Functional γδ T-lymphocyte defect associated with human immunodeficiency virus infections. Molecular Medicine 3, 60-71.
Wang, L., Kamath, A., Das, H., Li, L., and Bukowski, J. F. (2001). Antibacterial effect of human V gamma 2V delta 2 T cells in vivo. J Clin Invest 108(9), 1349-57.
Williamson, E. D., Eley, S. M., Stagg, A. J., Green, M., Russell, P., and Titball, R. W. (2000). A single dose sub-unit vaccine protects against pneumonic plague. Vaccine 19(4-5), 566-71.
Zheng, B. J., Chan, K. W., Im, S., Chua, D., Sham, J. S., Tin, P. C., He, Z. M., and Ng, M. H. (2001). Anti-tumor effects of human peripheral gammadelta T cells in a mouse tumor model. Int J Cancer 92(3), 421-5.

1. A method of protecting a person from disease arising from exposure to a biological pathogen comprising administering a composition comprising an amount of bisphosphonate sufficient to stimulate γ/δ T cells, wherein the bisphosphonate is risedronate.

2. The method according to claim 1, wherein the risedronate is administered in an amount from about 60 mg to about 120 mg.

3. The method according to claim 2, wherein the risedronate is administered by intravenous, intraperitoneal, oral, intralymphatic, or subcutaneous administration.

4. The method according to claim 1, wherein the composition further comprises a vaccine specific for the biological pathogen.

5. The method according to claim 1, further comprising administering a vaccine specific for the biological pathogen.

6. The method according to claim 1, further comprising administering IL-2 concurrently with the risedronate.

7. A method of stimulating γ/δ T cells to provide protection against infectious biological weapons, the method comprising administering a bisphosphonate in a sufficient amount to stimulate γ/δ T cells.

8. The method according to claim 7, wherein the bisphosphonate is administered after exposure to infectious biological weapons.

9. The method according to claim 7, wherein the bisphosphonate is administered before exposure to infectious biological weapons

10. A pharmaceutical composition comprising at least 40 mg of bisphosphonate in a pharmaceutically acceptable carrier, and a vaccine specific for a biological pathogen.

11. The pharmaceutical composition according to claim 10 comprising at least 120 mg of risedronate.

12. The pharmaceutical composition according to claim 10 further comprising IL-2.

13. A method of protecting a person against infection after exposure to a biological pathogen comprising administering risedronate immediately after exposure in an effective amount to stimulate γ/δ T cells.

14. A method of protecting a person against infection due to exposure to a biological pathogen comprising administering a dose of risedronate prior to coming into contact with a biological pathogen in an effective amount to stimulate γ/δ T cells.

15. The method according to claim 13 further comprising administering a vaccine specific for the biological pathogen

16. The method according to claim 14 further comprising administering a vaccine specific for the biological pathogen.
